(19) United States
(12) Patent Application Publication
(10) Pub. No.: US 2025/0352746 A1
(43) Pub. Date: Nov. 20, 2025

(54) INHALER

(71) Applicant: KT&G CORPORATION, Daejeon (KR)

(72) Inventors: Yongmi JUNG, Daejeon (KR); Moonwon KIM, Daejeon (KR); Jun Won SHIN, Daejeon (KR)

(73) Assignee: KT&G CORPORATION, Daejeon (KR)

(21) Appl. No.: 18/871,991

(22) PCT Filed: Jun. 2, 2023
(86) PCT No.: PCT/KR2023/007610

§ 371 Date: Dec. 5, 2024

(30) Foreign Application Priority Data
Sep. 19, 2022 (KR) 10-2022-0117659

(51) Int. Cl. A61M 15/00; A24F 42/20; A24F 42/60

(52) U.S. Cl. CPC A61M 15/004; A24F 42/20; A24F 42/60

(57) ABSTRACT

An inhaler for inhaling a functional material according to various embodiments comprises: a holder that includes an opening and an inner space communicating with the opening; piercing members which protrude into the inner space; and a functional material-accommodating member which is inserted along the longitudinal axis of the holder into the inner space of the holder from the opening and accommodates the functional material. One end of each of the piercing members is fixed to one side of the holder, and the tip of each of the piercing members faces the longitudinal axis.

### TECHNICAL FIELD

The following embodiments relate to an inhaler.

### BACKGROUND ART

Research has been conducted on an inhaler that may deliver a target substance directly to the lungs of a user. For example, Korean Patent Publication No. 10-2016-0065204 discloses a dry powder inhaler.

### DISCLOSURE OF THE INVENTION

### Technical Goals

An inhaler according to various embodiments may induce a vortex behavior of functional substance powder.

An inhaler according to various embodiments may induce smooth discharge of functional substance powder.

### Technical Solutions

An inhaler according to an embodiment may include a holder including an opening and an internal space that communicates with the opening, a piercing member protruding toward the internal space, and a functional substance accommodation member inserted from the opening into the internal space of the holder along a longitudinal axis of the holder and configured to accommodate the functional substance, wherein one end of the piercing member may be fixed to one side of the holder, and a cutting edge of the piercing member may be directed toward the longitudinal axis.

According to an embodiment, the piercing member may include a first piercing member and a second piercing member disposed spaced apart from the first piercing member along the longitudinal axis.

According to an embodiment, the first piercing member and the second piercing member may be disposed to face each other about the longitudinal axis.

According to an embodiment, a distance in a horizontal direction, which may be perpendicular to the longitudinal axis, between a cutting edge of the first piercing member and a cutting edge of the second piercing member may be less than a horizontal width of the functional substance accommodation member.

According to an embodiment, the functional substance accommodation member may include a stick configured to be insertable into the internal space of the holder and a capsule disposed in an inside of the stick and configured to accommodate the functional substance.

According to an embodiment, a marker for indicating a degree of insertion of the stick into the internal space may be provided in at least one of the stick or the holder.

According to an embodiment, the marker may indicate a first insertion state and a second insertion state of the stick, the first insertion state may be a position at which the capsule starts contacting the piercing member, and the second insertion state may be a position at which the stick reaches an end of the internal space.

According to an embodiment, the marker may indicate a third insertion state of the stick, and the third insertion state may be a state in which the stick is inserted into a position between the first insertion state and the second insertion state.

### Effects of the Invention

According to various embodiments, a vortex behavior of functional substance powder may be induced.

According to various embodiments, smooth discharge of functional substance powder may be performed by internal airflow introduced into an inhaler penetrating a center of a functional substance accommodation member.

The effects of an inhaler according to various embodiments are not limited to the above-mentioned effects, and other unmentioned effects can be clearly understood from the following description by one of ordinary skill in the art to which the present disclosure pertains.

### BRIEF DESCRIPTION OF DRAWINGS

FIG. 1 is a diagram illustrating an inhaler according to various embodiments.

FIG. 2 is a diagram illustrating an operating state of an inhaler according to various embodiments.

FIG. 3 is a diagram illustrating a puff state of an inhaler according to various embodiments.

### BEST MODE FOR CARRYING OUT THE INVENTION

The terms used in the embodiments are selected from among common terms that are currently widely used, in consideration of their function in the embodiments. However, the terms may become different according to an intention of one of ordinary skill in the art, a precedent, or the advent of new technology. Also, in particular cases, the terms are discretionally selected by the applicant of the disclosure, and the meaning of those terms will be described in detail in the corresponding part of the detailed description. Therefore, the terms used in the disclosure are not merely designations of the terms, but the terms are defined based on the meaning of the terms and content throughout the disclosure.

It will be understood that when a certain part “includes” a certain component, the part does not exclude another component but may further include another component, unless the context clearly dictates otherwise. Also, terms such as “unit,” “module,” etc., as used in the specification may refer to a part for processing at least one function or operation and may be implemented as hardware, software, or a combination of hardware and software.

As used herein, an expression such as “at least one of” that precedes listed components modifies not each of the listed components but all the components. For example, expressions “at least one of a, b, or c” and “at least one of a, b, and c” should be construed as referring to “a,” “b,” “c,” “a and b,” “a and c,” “b and c,” or “a, b, and c.”

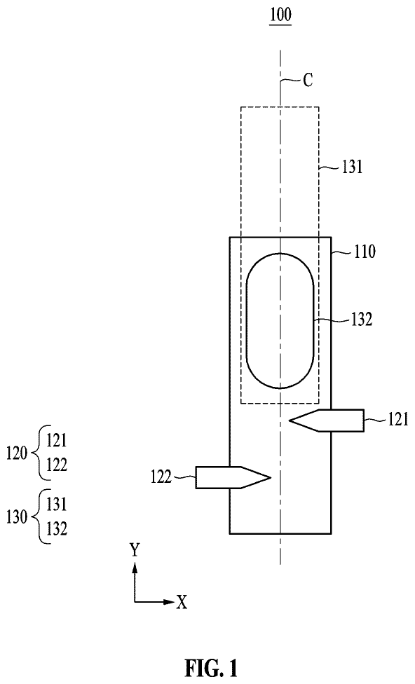

FIG. 1 is a diagram illustrating an inhaler 100 according to an embodiment.

The inhaler 100 according to an embodiment may deliver a functional substance to a user. For example, the inhaler 100 according to an embodiment may be composed of an inhaler that delivers a dried pharmacological substance or nicotine to the lungs of a user in a form of an aerosol.

Referring to FIG. 1, the inhaler 100 according to an embodiment may include a holder 110, a piercing member 120, and a functional substance accommodation member 130.

In an embodiment, the holder 110 may be configured in a cylindrical shape with an internal space, and the internal space may be formed to a size into which the functional substance accommodation member 130, which is described below, may be inserted. An opening may be formed in a side of the holder 110, wherein the functional substance accommodation member 130 is inserted through the opening, and the opening and the internal space may be connected to each other.

In an embodiment, the piercing member 120 may include a first piercing member 121 and a second piercing member 122. One side of the first piercing member 121 may be fixed to an inside of the holder 110, and the other side of the first piercing member 121 may be configured as a cutting edge protruding toward a longitudinal axis C of the holder 110. One side of the second piercing member 122 may be fixed to the inside of the holder 110, and the other side of the second piercing member 122 may be configured as a cutting edge protruding toward the longitudinal axis C of the holder 110.

The first piercing member 121 and the second piercing member 122 may be disposed to be spaced apart in a direction parallel to the longitudinal axis C. For example, the first piercing member 121 and the second piercing member 122 may be disposed to be spaced apart from each other in a +Y direction of FIG. 1.

In addition, the first piercing member 121 and the second piercing member 122 may be disposed to face each other about the longitudinal axis C. For example, the first piercing member 121 and the second piercing member 122 may be disposed to be spaced apart from each other in a +X direction of FIG. 1 with respect to the longitudinal axis C.

In an embodiment, the functional substance accommodation member 130 may include a stick 131 and a capsule 132. For example, the stick 131 may be configured in a cylindrical shape that may be inserted into the internal space of the holder 110, and the capsule 132 may be disposed in an inside of the stick 131.

An area of the functional substance accommodation member 130, in which the capsule 132 may be disposed, may be set so that the capsule 132 may be at a position overlapping the first piercing member 121 or the second piercing member 122 when the stick 131 reaches an end of the internal space of the holder 110.

In an example, the capsule 132 may accommodate a functional substance. The functional substance may include, for example, at least one of nicotine, theanine, caffeine, taurine, a pharmacological substance, or a mixture thereof. The functional substance may be in a form of fine granules or dry powder.

In an example, an outer shell of the capsule 132 may be made of a material that may be pierced or cut by the first piercing member 121 or the second piercing member 122.

In an embodiment, the first piercing member 121 and the second piercing member 122 may be fixed to the holder 110 at positions at which a horizontal distance (e.g., a distance in a +X direction of FIG. 1) between the first piercing member 121 and the second piercing member 122 is less than an outer diameter of the capsule 132. Here, a horizontal direction is a direction perpendicular to the longitudinal axis C.

Since a horizontal distance between the cutting edge of the first piercing member 121 and the cutting edge of the second piercing member 122 may be less than a horizontal width of the capsule 132, when the stick 131 is inserted into the internal space of the holder 110, at least a portion of the capsule 132 may be cut by the first piercing member 121 or the second piercing member 122 and thus the functional substance in the capsule 132 may be exposed to an outside of the capsule 132.

In an embodiment, a marker (not shown) for indicating a degree of insertion of the stick 131 into the internal space may be provided in at least one of the holder 110 or the stick 131 of the functional substance accommodation member 130. When an open area in a longitudinal slit shape is formed in the capsule 132, a size of the slit may be indirectly measured by the marker, and the size of the slit may be set to be proportional to an amount of a puff of the functional substance.

In an embodiment, the marker may indicate a first insertion state and a second insertion state of the stick 131. The first insertion state may be a position at which the capsule 132 starts contacting the piercing member 120, and the second insertion state may be a position at which the stick 131 reaches the end of the internal space.

In addition, the marker may indicate a third insertion state of the stick 131, and the third insertion state may be a state in which the stick 131 is inserted into a position between the first insertion state and the second insertion state.

For example, as the stick 131 is inserted into the internal space of the holder 110 and moves toward the end of the internal space, an end of the stick 131 may be the first to be cut by the first piercing member 121 or the second piercing member 122. Here, it may be confirmed that the first piercing member 121 may start cutting the capsule 132 when the stick 131 reaches the first insertion state indicated on the marker. The user may adjust a desired amount of a puff by further moving the stick 131 toward the third insertion state indicated on the marker. Alternatively, the user may set the desired amount of a puff to a maximum amount of a puff by moving the stick 131 toward the second insertion state indicated on the marker. For example, according to the number of puffs, the amount of a puff of the stick 131 may be adjusted to maintain the third insertion state at an initial puff and maintain the second insertion state at subsequent puffs.

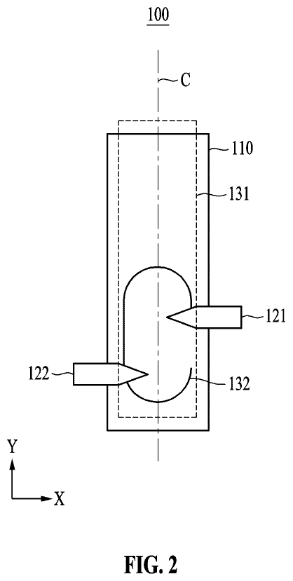

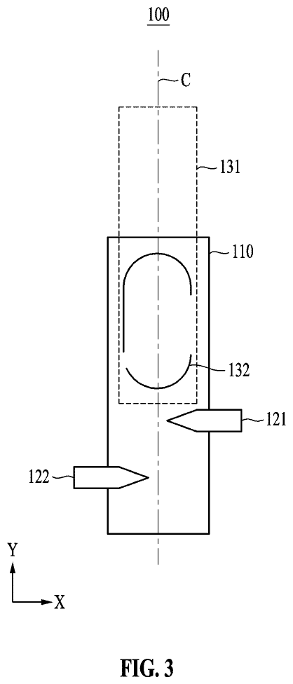

FIG. 2 and FIG. 3 are diagrams illustrating an operating state of the inhaler 100 according to an embodiment. FIG. 2 illustrates a state in which the functional substance accommodation member 130 is inserted into the internal space of the holder 110, and FIG. 3 illustrates a state in which the functional substance may transfer while a portion of the capsule 132 is cut.

Referring to FIG. 2, the functional substance accommodation member 130 may be inserted into the internal space of the holder 110. Here, at least a portion of the capsule 132 may be cut by the first piercing member 121 or the second piercing member 122 and thus a longitudinal slit may be formed in the capsule 132.

As described above, the stick 131 may be inserted into the internal space to one of the second insertion state or the third insertion state by the marker and thus a degree of opening of the slit may be adjusted.

A slit-shaped open area by the first piercing member 121 and a slit-shaped open area by the second piercing member 122 may be formed in the capsule 132. Since the open areas may be formed spaced apart along the longitudinal axis C, airflow passing through the open areas may pass through a central area of the capsule 132.

Referring to FIG. 3, at least a portion of the functional substance accommodation member 130 may be separated from the holder 110. Here, the functional substance accommodation member 130 may be completely separated from the holder 110, but the functional substance may transfer while the capsule 132 is inserted in the holder 110. In this case, separation of the functional substance may effectively be prevented compared to a case in which the functional substance accommodation member 130 is completely separated from the holder 110, and thus, unnecessary leakage of the functional substance may be prevented.

When the user bites one end of the stick 131 and makes an inhaling motion while the open area is formed in the capsule 132, external air may flow into the internal space of the holder 110 through an airflow hole (not shown) formed in the holder 110, and the air may flow into the capsule 132 through the open area formed by the second piercing member 122. Subsequently, the air may escape through the open area formed by the first piercing member 121 and reach the user, in which case the functional substance in the capsule 132 may also be transferred to the user. Due to eccentric positions of the open areas, the air may pass through a central part of the capsule 132 and, at the same time, a vortex behavior of the functional substance may be induced. The vortex behavior may induce smooth movement of the functional substance, and accordingly, discharge of the functional substance may be performed more smoothly.

In an example, when the capsule 132 is disposed in the stick 131 in an unfixed state, air movement (airflow) generated due to an eccentric position of the open area may induce the rotation of the capsule 132. Through this, the discharge of the functional substance may be performed more smoothly.

The description of the above-described embodiments is only illustrative, and one of ordinary skill in the art will understand that various modifications and other equivalent embodiments are possible therefrom. Therefore, the true scope of protection of the present disclosure should be determined by the appended claims, and all differences within the equivalent scope of what is stated in the claims should be interpreted as being included in the scope of protection determined by the claims.

1. An inhaler for inhaling a functional substance, the inhaler comprising:
a holder comprising an opening and an internal space that communicates with the opening;
a piercing member protruding toward the internal space; and
a functional substance accommodation member inserted from the opening into the internal space of the holder along a longitudinal axis of the holder and configured to accommodate the functional substance,
wherein one end of the piercing member is fixed to one side of the holder, and a cutting edge of the piercing member is directed toward the longitudinal axis.

2. The inhaler of claim 1, wherein the piercing member comprises:
a first piercing member; and
a second piercing member disposed spaced apart from the first piercing member along the longitudinal axis.

3. The inhaler of claim 2, wherein the first piercing member and the second piercing member are disposed to face each other about the longitudinal axis.

4. The inhaler of claim 2, wherein a distance in a horizontal direction, which is perpendicular to the longitudinal axis, between a cutting edge of the first piercing member and a cutting edge of the second piercing member is less than a horizontal width of the functional substance accommodation member.

5. The inhaler of claim 1, wherein the functional substance accommodation member comprises:
a stick configured to be insertable into the internal space of the holder; and
a capsule disposed in an inside of the stick and configured to accommodate the functional substance.

6. The inhaler of claim 5, wherein a marker for indicating a degree of insertion of the stick into the internal space is provided in at least one of the stick or the holder.

7. The inhaler of claim 6,
wherein the marker indicates a first insertion state and a second insertion state of the stick, and
wherein the first insertion state is a position at which the capsule starts contacting the piercing member, and the second insertion state is a position at which the stick reaches an end of the internal space.

8. The inhaler of claim 7,
wherein the marker indicates a third insertion state of the stick, and
wherein the third insertion state is a state in which the stick is inserted into a position between the first insertion state and the second insertion state.
